(19) United States
(12) Patent Application Publication
(10) Pub. No.: US 2023/0158274 A1
(43) Pub. Date: May 25, 2023

(54) Protection Against Unauthorized Access to Intravenous Catheter While Infusion In-Progress

(71) Applicant: PICCGuard LLC, Sheboygan, WI (US)

(72) Inventors: Katherine Justus, Sheboygan, WI (US); Jesse Justus, Sheboygan, WI (US); Michael Justus, Beloit, WI (US)

(21) Appl. No.: 18/058,452

(22) Filed: Nov. 23, 2022

Related U.S. Application Data
(60) Provisional application No. 63/282,922, filed on Nov. 24, 2021.

(51) Int. Cl. A61M 25/02

(52) U.S. Cl. CPC A61M 25/02

(57) ABSTRACT

A temporary catheter security device for temporarily locking an infusion catheter coupling while an infusion is in-progress. This catheter security device is designed to prevent, discourage, or detect unauthorized access to this catheter coupling. The catheter security device could have an enclosure design or a bag-type design. The catheter security device has openings where the infusion tubings exit. These openings are sufficiently wide to avoid obstruction of the fluid infusion.

### TECHNICAL FIELD

This invention relates generally to intravenous catheters, and more particularly to accessory devices to prevent unauthorized use thereof for self-administering illicit drugs.

### BACKGROUND

Medical treatment often involves the infusion of medications through an intravenous (IV) catheter. However, many drug abusers see this IV catheter as a convenient access for self-administering illicit drugs. While the medication infusion is in-progress, and when medical personnel are not watching, they may covertly detach the IV catheter from the medication infusion and quickly use the IV catheter to self-administer an unauthorized drug. Thus, there is a need for making IV catheters more resistant to unauthorized use, while allowing an infusion to proceed.

### SUMMARY

This invention is used for patients who have a venous catheter inserted into a venous blood vessel. The patient receives a medical fluid through this venous catheter. Examples of medical fluids include cancer chemotherapy, antibiotic/antifungal medications, nutrients, immunoglobulins, etc. This invention could be used with any of various types of venous catheters such as standard peripheral venous catheters (i.e. IV lines), or long-term indwelling catheters such as central venous access catheters, hemodialysis catheters, peripherally inserted central catheters (PICC), or midline catheters.

The medical fluid is administered through an infusion line (e.g. a conventional IV line connected to an IV fluid bag), which comprises an infusion tubing. The infusion line has a connector (e.g. a luer-type adaptor); and the venous catheter has a counterpart connector (e.g. a luer fitting that screws into a luer adaptor). The connector for the infusion line is coupled to the counterpart connector for the patient's venous catheter to create an infusion catheter coupling. This invention is designed to prevent, discourage, or detect unauthorized access to this catheter coupling.

In one aspect, this invention is a temporary catheter security device for temporarily locking an infusion catheter coupling while an infusion is in-progress. The infusion catheter coupling is placed inside the catheter security device and locked therein to prevent unauthorized access. Enclosure Design: In one embodiment, the security device comprises a cover piece and a base piece that are designed to mate with each other to form an enclosure. The security device has an open configuration and a closed configuration. When the cover piece and base piece are not mated, the security device is in open configuration.

The catheter security device has a locking mechanism to lock the device in closed configuration. For the locking mechanism, there is a locking part on the cover piece and a locking counterpart on the base piece. The locking part and the locking counterpart engage with each other as the locking mechanism. When the cover piece and base piece are locked together in closed configuration, they form an enclosure within. The locking mechanism is designed to be breakable by manual force with or without a hand tool (with no additional force provided by electric power, such as a motor). See more details below about breaking the locking mechanism.

The catheter security device could have any suitable shape for holding an infusion catheter coupling therein. For example, the device could have a rectangular or cylindrical shape. The catheter security device could have any suitable dimensions. In some cases, the enclosure (formed by the cover piece and a base piece) has a length in the range of 5-15 cm. In some cases, the enclosure has a width in the range of 1-5 cm. In some cases, the enclosure has a depth in the range of 1-5 cm.

The enclosure (formed by the locking of the cover piece to the base piece) has a first opening at one end and a second opening at another end. These openings are where the infusion tubing (of the infusion line) and the catheter tubing (of the venous catheter) exit from the enclosure. The catheter security device is designed to allow an infusion to continue while the infusion catheter coupling is enclosed therein. Thus, the tubings should not be pinched closed. As such, the catheter security device has openings that sufficiently wide to avoid obstruction of the medical fluid infusion. In some cases, the first and second openings each independently have a width in the range of 1.0-6.0 mm wide; and in some cases, width in the range of 2.0-4.0 mm wide; and

In some embodiments, the catheter security device is a clamshell design with the cover piece being a first shell and the base piece being a second shell, and the device further comprises a hinged spine connecting the first shell and the second shell. In some cases, the first shell comprises a first pair of notches and the second shell comprises a second pair of notches. The first pair of notches and the second pair of notches are configured to match to form the openings for the enclosure.

In some embodiments, the catheter security device is a box design with the cover piece being a top lid and the base piece being a bottom box having a compartment. The top lid is configured to be fitted over the bottom box. In some cases, the bottom box has a pair of notches that form openings for the enclosure. The top lid may or may not have a counterpart notch.

Method of Use: In another aspect, this invention is a method of infusing a medical fluid through a patient's venous catheter while locking the catheter coupling inside an enclosure. The above-described catheter security device could be used. The catheter coupling is placed onto the base piece. The cover piece is mated to the base piece so that the catheter security device is in closed configuration. The locking part and the locking counterpart are engaged to lock the catheter security device in closed configuration and form the enclosure. With this, the catheter coupling is contained in the enclosure and locked therein. The catheter tubing exits out of the enclosure at the one opening. The infusion tubing exits out of the enclosure at the other opening. The medical fluid is infused through the infusion line and into the venous catheter while the catheter security device is locked in closed configuration. The infusion may be started before or after implementing the security device.

When the infusion is completed, administration of the medical fluid is stopped and the catheter coupling is ready to be released. This is done by permanently breaking the locking mechanism on the security device. There are a variety of ways to permanently break the locking mechanism, such as cutting a part of the locking mechanism (e.g. with scissors or cutting forceps) or manually deforming the locking mechanism with a tool (twisting off with pliers or forceps clamp). The catheter coupling is removed from the security device, which may be performed before or after stopping administration of the medical fluid. Further post-infusion steps may include uncoupling the infusion line from the venous catheter.

Bag-Type Design: In another embodiment, the security device comprises a pouch having a main body and one or more detachment portions. The detachment portion is demarcated from the main body by an indicia line or dividing line. The indicia line may be any type of marking on the pouch, such as printed ink lines or scratches made into the surface. The dividing line may be any type of structural alteration to the pouch to facilitate breaking off the detachment portion. Examples of dividing lines include grooves, scorings, perforations, etc.

The security device further comprises one or more fasteners on a first side of the pouch, and one or more fasteners on a second side of the pouch. Adjacent or between the fasteners, there is a first opening at the first side of the pouch, and a second opening at the second side of the pouch. The catheter security device is designed to allow the infusion to continue while the catheter coupling is locked therein. As such, the openings are sufficiently wide to avoid obstruction of the medical fluid infusion. In some cases, the first and second openings each independently have a width in the range of 1.0-7.0 mm wide; and in some cases, in the range of 2.0-5.0 mm wide.

Method of Use: In another aspect, this invention is a method of infusing a medical fluid through a patient's venous catheter while locking the catheter coupling inside a bag-type security device. The catheter coupling is placed inside the pouch. This could be done by making the catheter coupling outside of the pouch (e.g. connecting the venous catheter to the infusion line outside the pouch), and after the catheter coupling is made, putting the catheter coupling inside the pouch.

The fasteners on both sides are engaged to lock the pouch. With this, the catheter coupling is contained in the pouch and locked therein. The catheter tubing exits out of the pouch at the one opening. The infusion tubing exits out of the pouch at the other opening. The medical fluid is infused through the infusion line and into the venous catheter while the catheter security device is locked. The infusion may be started before or after implementing the security device.

When the infusion is completed, administration of the medical fluid is stopped and the catheter coupling is ready to be released. This is done by detaching the detachment portion of the pouch. Detaching may be performed in any suitable manner, such as cutting, tearing, splitting, cracking, fracturing, etc. The catheter coupling is removed from the security device, which may be performed before or after stopping administration of the medical fluid. Further post-infusion steps may include uncoupling the infusion line from the venous catheter.

### BRIEF DESCRIPTION OF THE DRAWINGS

FIG. 1 shows a conventional coupling of a PICC line to an IV infusion line.

FIG. 2 shows an example of a temporary catheter security device.

FIGS. 3-5 show the security device in closed configuration to form an enclosure containing the catheter coupling. FIG. 3 shows a top view; FIG. 4 shows a perspective view from the IV line side; FIG. 5 shows a different perspective view from the PICC line side.

FIG. 6 shows an alternate configuration for using the same security device.

FIG. 7 shows a different locking mechanism that could be used.

FIG. 8 shows another locking mechanism that could be used.

FIGS. 9A-C show an alternate box-type design for the security device. FIG. 9A is a side view; FIG. 9B is an end-on view; FIG. 9C shows the locking parts engaged.

FIGS. 10A and 10B show a bag-type security device that comprises a plastic pouch. FIG. 10A shows a top view; FIG. 10B shows a left side end-on view.

FIG. 11 shows the bag-type security device when the infusion is finished.

FIG. 12 shows a different design for a bag-type security device.

### DETAILED DESCRIPTION OF EXAMPLE EMBODIMENTS

To assist in understanding the invention, reference is made to the accompanying drawings to show by way of illustration specific embodiments in which the invention may be practiced. The drawings herein are not necessarily made to scale or actual proportions. For example, lengths and widths of the components may be adjusted to accommodate the page size.

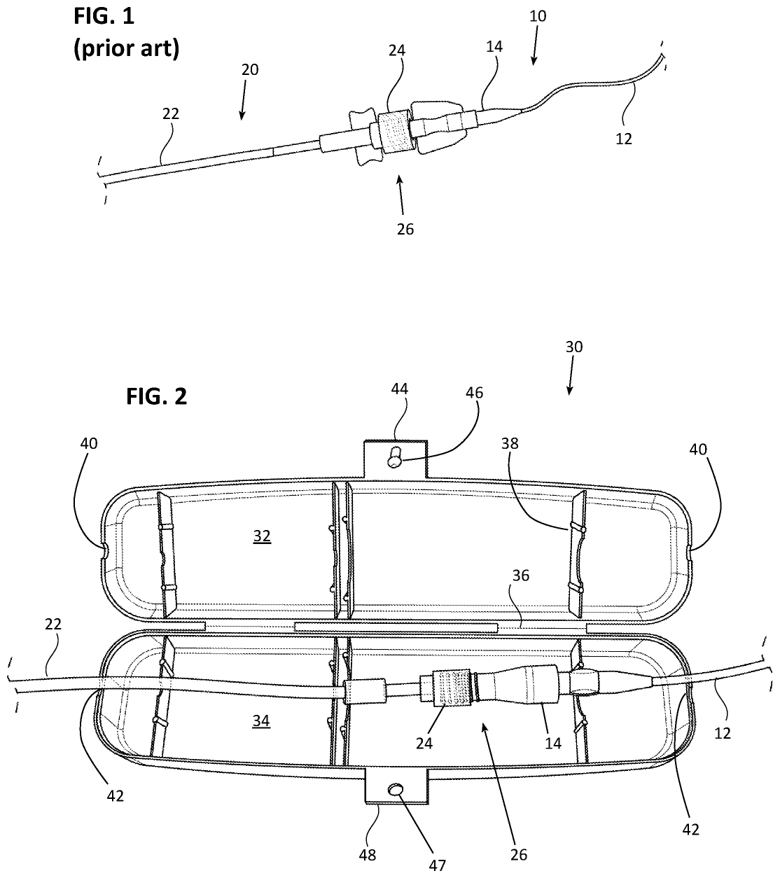

FIG. 1 shows a conventional coupling of a PICC (peripherally inserted central catheter) line 10 to an IV line (intravenous) line 20. IV line 20 has a tubing 22 and a luer-type connector 24. PICC line 10 has a tubing 12 and a luer-fitting 14. The engagement of luer-type connector 24 of IV line 20 to luer-fitting 14 of PICC line 10 makes a catheter coupling 26. IV line 20 is attached to a source of medication fluid (e.g. an infusion bag) and PICC line 10 has already been inserted into the patient's vein. Thus, the medical fluid flows through IV line 20, into PICC line 10, and then into the patient's vein.

FIG. 2 shows an example of a temporary catheter security device 30 (perspective view). Security device 30 is a clamshell design with a top shell 32 and a bottom shell 34. Top shell 32 and bottom shell 34 are connected by a flexible hinge 36 that runs along the spine of the back side. Here, security device 30 is shown in open configuration with the catheter coupling 26 laid onto the bottom compartment of bottom shell 34. Top shell 32 and bottom shell 34 and have ridges 38 to support the various parts of the infusion setup.

Top shell 32 has a pair of small half-circle notches 40. Likewise, bottom shell 34 has a pair of small half-circle notches 42. IV tubing 22 and PICC line tubing 12 are set into notches 42 of the bottom shell 34. As will be seen later, these notches 40 and 42 will make exit holes where IV tubing 22 and PICC line tubing 12 exit.

Security device 30 has a locking mechanism. There is a top locking lip 44 at the front side edge of top shell 32. At the bottom side of top locking lip 44, there is a barbed protrusion 46 that works as a snap lock. There is a counterpart bottom locking lip 48 at the front side edge of bottom shell 34. Bottom locking lip 48 has a small hole 47 for receiving barbed protrusion 46.

Top locking lip 44 and bottom locking lip 48 are designed to mate to form a snap lock for security device 30.

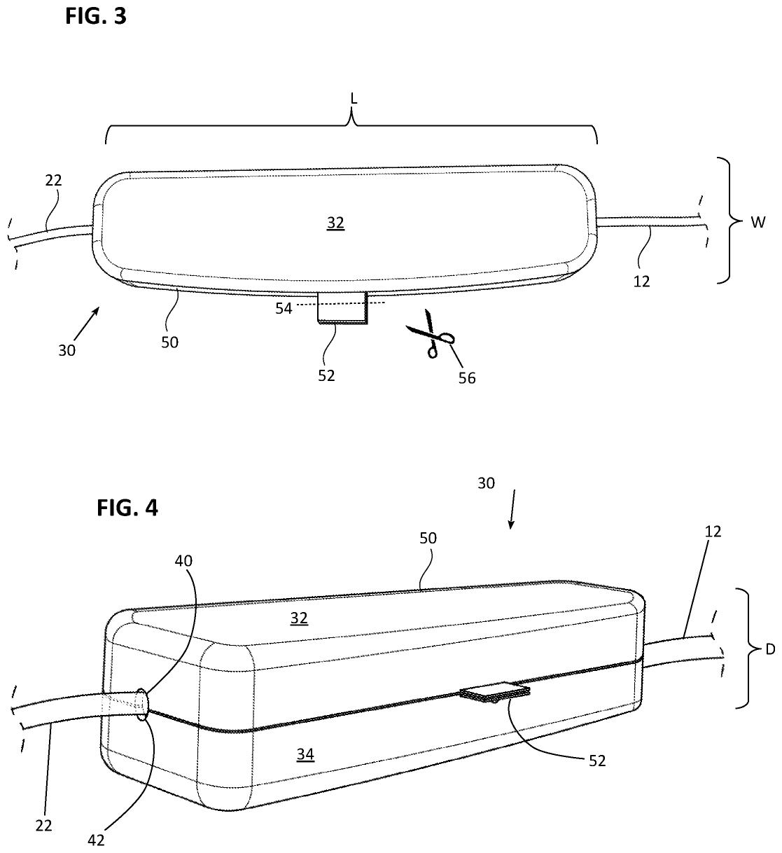

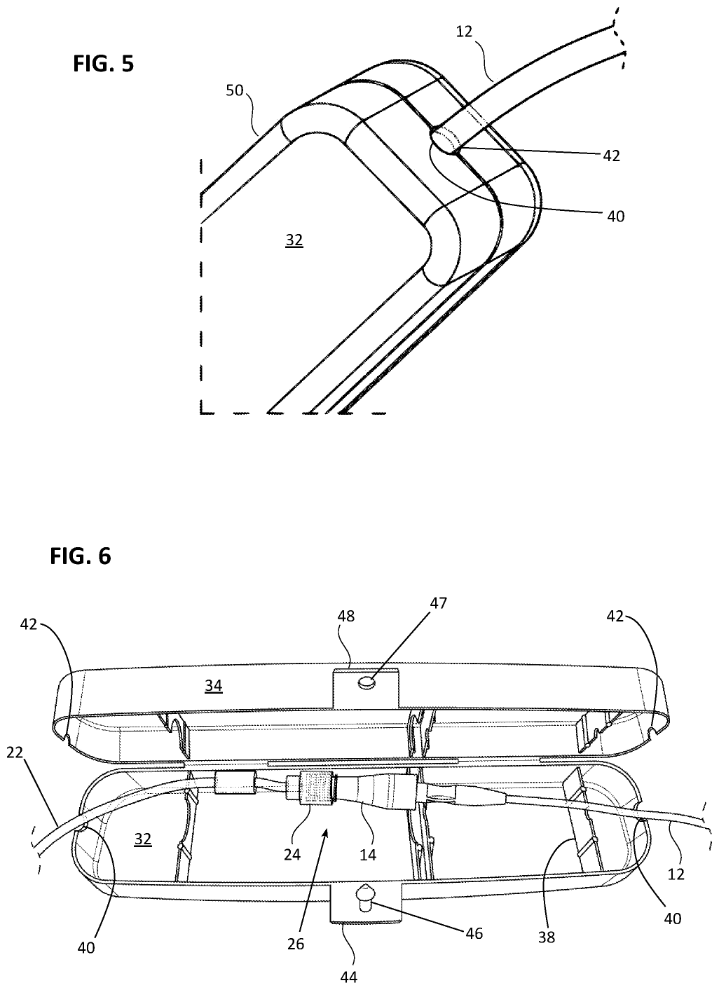

FIGS. 3-5 show security device 30 in closed configuration to form an enclosure 50 containing catheter coupling 26 (hidden inside). FIG. 3 shows a top view; FIG. 4 shows a perspective view from the IV line side; FIG. 5 shows a different perspective view from the PICC line side. FIG. 3 shows top locking lip 44 and bottom locking lip 48 mated to form a snap lock 52. Dashed line 54 indicates where the snap lock 52 can be cut by scissor 56 or knife to sever snap lock 52 and allow opening of enclosure 50 to expose catheter coupling 26. Dashed line 54 could also be an actual indicia marking on top locking lip 44 (e.g. a line drawn on it, accompanied by print stating “cut here”). In closed configuration for security device 10, enclosure 50 has a length (L) of about 9 cm, a width (W) of about 2 cm, and a depth (D) of about 2 cm.

FIG. 4 shows how the notch 40 of top shell 32 and counterpart notch 42 of bottom shell 34 match to form an exit hole for IV tubing 22. At the other end of enclosure 50, FIG. 5 shows how the notch 40 of top shell 32 and counterpart notch 42 of bottom shell 34 match to form an exit hole for PICC line tubing 12. The exit holes have a width of about 3 mm. With catheter coupling 26 locked inside security device 30, the patient is prohibited from unauthorized access to the catheter coupling 26.

FIG. 6 shows an alternate configurations in which the same security device 30 is used, but the catheter coupling 26 is laid onto the top compartment of top shell 32 (instead of on bottom shell 34). In this instance, top shell 32 is the base piece and bottom shell 34 is the cover piece. Otherwise, security device 30 operates in the same way as above. Note that in FIGS. 4-6, IV tubing 22 and PICC line tubing 12 are not pinched closed. That is, the exits holes at both ends of enclosure 50 are wide enough to freely accommodate IV tubing 22 and PICC line tubing 12. Thus, the medical fluid can continue flowing through IV line 20, into the PICC line 10, and then into the patient's vein, even with security device 30 locked shut. The infusion may be started before or after security device 30 is locked shut.

When the infusion is finished, snap lock 52 is cut in the manner shown in FIG. 3 above. This exposes catheter coupling 26, which is removed from security device 30. Thus, security device 30 is only for temporary use during the infusion procedure. After the infusion procedure is complete and security device 30 is removed, a more long-term catheter enclosure device could be used, such as those described in U.S. Patent Application Pub. No. 2019/0381305 (date 19 Dec. 2019 to Justus), or US 2020/0391001 (date 17 Dec. 2020 to Justus et al.); or U.S. Pat. No. 8,858,505 (date 14 Oct. 2014 to Justus et al.).

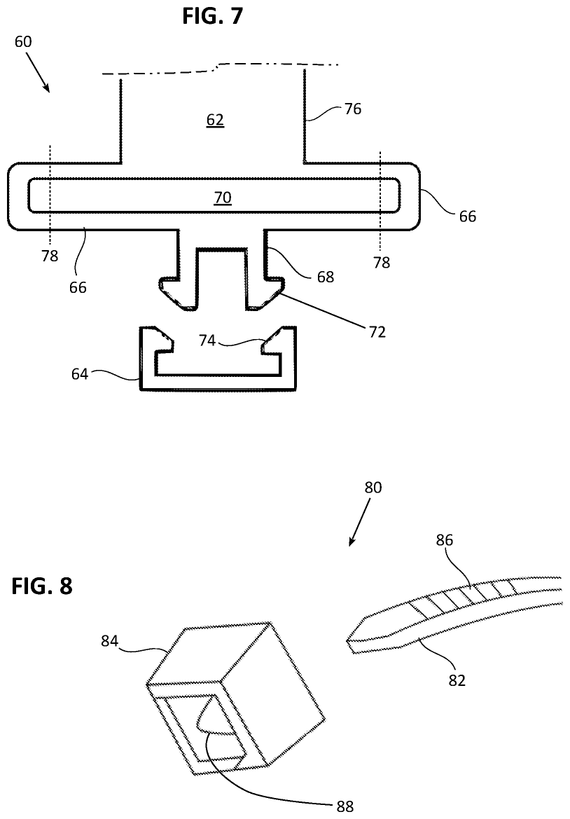

FIG. 7 shows a different locking mechanism that could be used. In locking mechanism 60, there is an anchor part 62 of the locking mechanism, which can be considered as having two sections. There is an upper section 76 and a lower section 68. These two sections are connected by bridge segments 66 on each lateral side. The bridge segments 66 follow a path that creates a void window 70. Lower section 68 of the anchor part 62 has two flexible arms 72 that are designed to flex inward and then snap outward.

The bottom part of locking mechanism 60 is a slot piece 64, which is designed to receive anchor part 62 for a snap lock mechanism. Slot piece 64 has two hooks 74 for engaging with the two flexible arms 72 of the anchor part 62. As slot piece 64 moves downward, flexible arms 72 slides against hooks 74 and flex inward. When fully inserted into slot piece 64, flexible arms 72 snap outward to become retained within slot piece 64. This locks anchor part 62 and slot piece 64 together. Dashed lines 78 indicates where bridge segments 66 can be cut by a scissor or knife. This severs the connection between upper section 76 of anchor part 62 and slot piece 64. This allows opening of the enclosure to expose the catheter coupling so that it can be removed.

FIG. 8 shows another locking mechanism that could be used. In locking mechanism 80, there is a pawl spear 82 and a slot 84. Pawl spear 82 has a series of teeth 86. Slot 84 has a ratchet 88. As pawl spear 82 is inserted into slot 84, teeth 86 get caught in ratchet 88. This pawl-ratchet mechanism locks pawl spear 82 and slot 84 together.

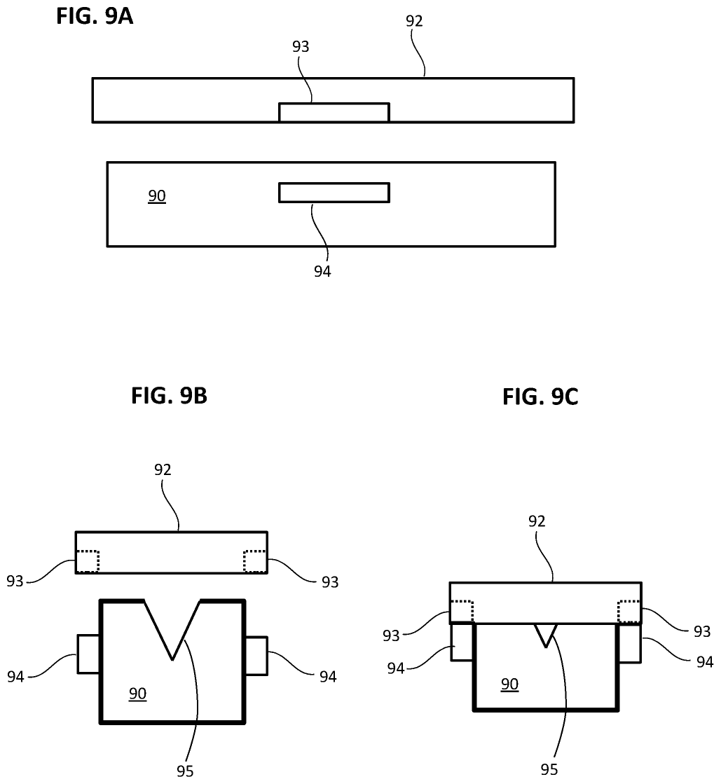

The catheter security device does not necessarily have to be a clamshell design. FIGS. 9A-C show an alternate design in which there is a bottom box 90 and a top lid 92. FIG. 9A is a side view showing when the bottom box 90 is open for receiving the catheter coupling in the compartment therein. There is no hinge connecting bottom box 90 and top lid 92. Top lid 92 has upper locking parts 93 on each side; bottom box 90 has lower locking parts 94 on each side. Locking parts 93 and 94 are designed to engage and make a secure lock.

FIG. 9B is an end-on view showing a V-shaped notch 95 at each end wall of bottom box 90. The IV line and the PICC line are put into V-shaped notches 95 at each end. After the catheter coupling and infusion lines are put in place, top lid 92 is fitted onto bottom box 90. As shown in FIG. 9C (end-on view), locking parts 93 and 94 engage to make a secure lock, thereby making an enclosure for the catheter coupling and infusion lines. The infusion lines exit bottom box 90 out of notches 95, which are partially covered by top lid 92.

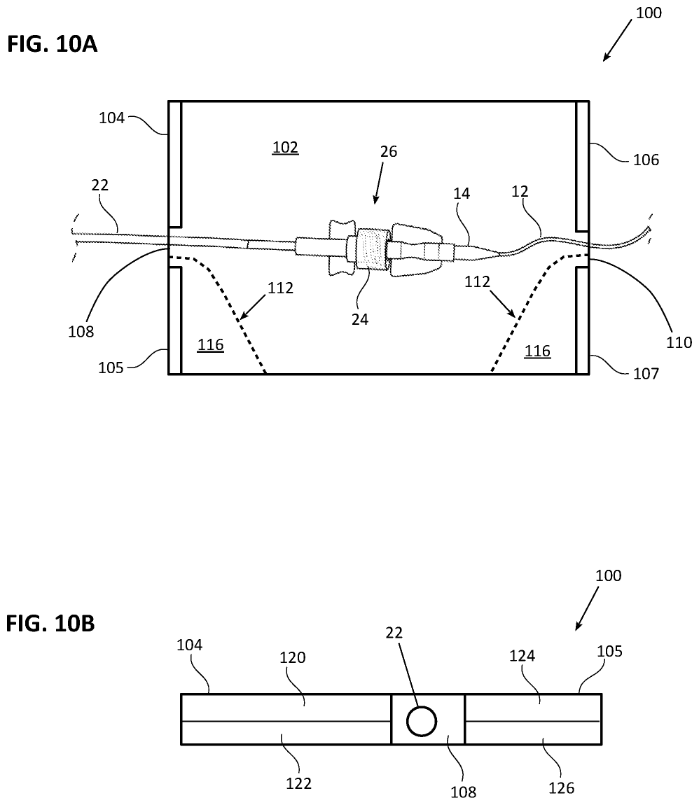

The catheter security device does not have to be a rigid box-like design. FIGS. 10A and 10B show a bag-type security device 100 that comprises a transparent, rectangular-shaped plastic pouch that is of sufficient size to contain catheter coupling 24 held inside. The pouch has a main body 102 and two detachment portions 116. There are two tear-off lines 112 that demarcate main body 102 of the pouch from the two detachment portions 116. The pouch is fully open on each side (left/right) so that catheter coupling 24, IV line tubing 22, and PICC line tubing can be placed inside.

On each side (left/right), security device 100 has a set of fasteners to lock the catheter coupling 24 inside. On the left side, there are fasteners 104 and 105. On the right side, there are fasteners 106 and 107. Any suitable type of fastening mechanism may be used for security device 100, such as those described in U.S. Patent Application Pub. No. US 2019/0381305 (19 Dec. 2019), which is incorporated by reference herein.

With catheter coupling 24 placed inside the pouch, all of fasteners 104, 105, 106, and 107 are fastened to close-off the pouch on the left and right sides. Note that there is a small opening 108 between the two fasteners 104 and 105. This is where the IV line tubing 22 exits. Note that there is a small opening 110 between the two fasteners 106 and 106. This is where the catheter line tubing 12 exits. In use, the catheter coupling 24 is made outside of the pouch, and after the catheter coupling 24 is made, it is and then pulled into the pouch compartment.

FIG. 10B shows a left side end-on view of security device 100. As seen here, fastener 104 has two parts 120 and 122 that lock together. Likewise, fastener 105 has two parts 124 and 126 that lock together. Opening 108 is where IV line tubing 22 exits. Thus, security device 100 is designed to allow the infusion is continue while the catheter coupling 24 is locked inside security device 100.

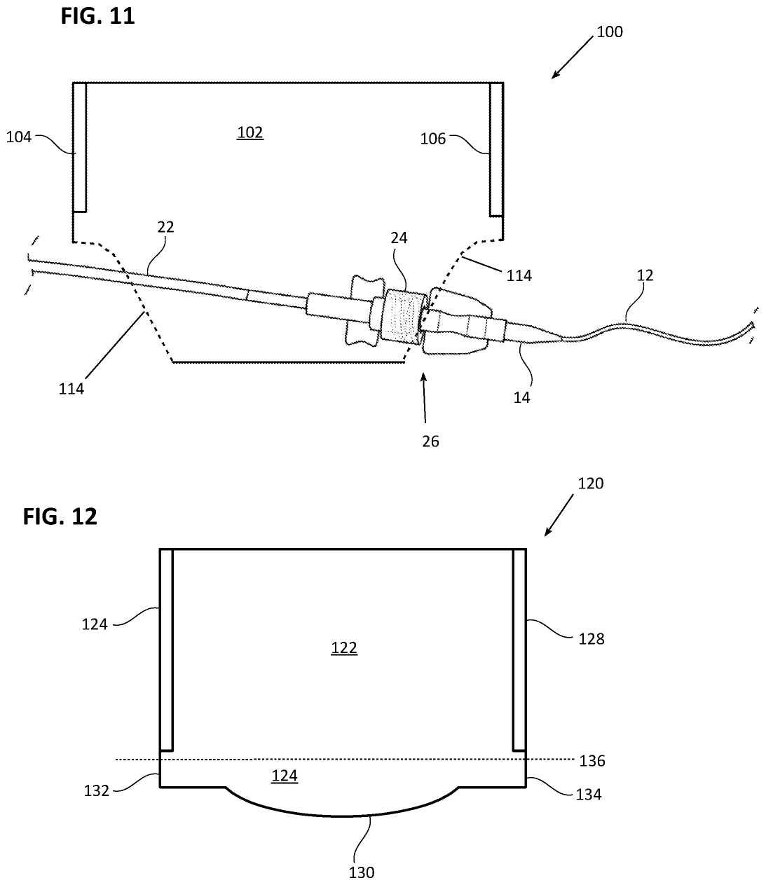

FIG. 11 shows when the infusion is finished. To remove catheter coupling 24, security device 100 is torn along tear lines 112 and detachment portions 116 are detached. This leaves openings 114 where the pouch is open. From openings 114, catheter coupling 24 can be pulled out of the pouch. With the infusion completed, the catheter coupling 24 is uncoupled and the luer fitting of the PICC line is capped.

FIG. 12 shows a different design for a bag-type security device in which there is only a single tear line. Here, security device 120 comprises a transparent plastic pouch with a main body 122 and a detachment part 124. The pouch has a bulge 130 to give extra room for accommodating the catheter coupling. Tear-off line 136 demarcates main body 122 of the pouch from detachment part 124. On the left side, there is a single fastener 124. On the right side, there is a single fastener 128. Fasteners 124 and 128 do not extend along the entire side to allow openings 132 and 134 on each side where the infusion tubings can exit. When the infusion is finished, security device 120 is torn along tear line 126 and detachment portion 124 is detached. This allows the catheter coupling to be removed from security device 120.

The descriptions and examples given herein are intended merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

The terms “first, second, etc.” with respect to elements are being used herein only to distinguish one element from another element. But these are not intended to limit the elements in an ordinal fashion, such as defining the order, position, or priority of the elements. For example, a first element could be alternately be called a second element, and, similarly, a second element could alternately be called a first element, without departing from the scope of the present invention. The first element and the second elements are not the same element. The terms “first, second, etc.” may refer to different elements in different embodiments.

1. A catheter security device comprising:
a cover piece;
a base piece, wherein the catheter security device is in an open configuration when the cover piece and base piece are not mated, and wherein the catheter security device is in a closed configuration when the cover piece and base piece are mated;
a locking part on the cover piece;
a locking counterpart on the base piece, wherein the locking part and the locking counterpart engage as a locking mechanism to lock the catheter security device in closed configuration;
wherein the closed configuration forms an enclosure that has a first opening at one side of the enclosure and a second opening at another side of the enclosure;
wherein the first and second openings each, independently, have a width in the range of 1.0-6.0 mm.

2. The device of claim 1, wherein the enclosure has a length in the range of 5-15 cm and a width in the range of 1-5 cm.

3. The device of claim 1, wherein the first and second openings each, independently, have a width in the range of 2.0 to 4.0 mm.

4. The device of claim 1, wherein the catheter security device is a clamshell design with the cover piece being a first shell and the base piece being a second shell, and the device further comprises a hinged spine connecting the first shell and the second shell.

5. The device of claim 4, wherein the first shell comprises a first pair of notches and the second shell comprises a second pair of notches, wherein the first pair of notches and the second pair of notches are configured to match to form the openings for the enclosure.

6. The device of claim 1, wherein the catheter security device is a box design with the cover piece being a top lid and the base piece being a bottom box having a compartment, and wherein the top lid is configured to be fitted over the bottom box.

7. The device of claim 6, wherein the bottom box has a pair of notches that form an opening for the enclosure.

8. The device of claim 7, wherein the top lid does not have a counterpart notch.

9. The device of claim 1, wherein the locking mechanism is a snap lock.

10. The device of claim 1, wherein the locking mechanism has indicia indicating where cutting of the locking mechanism should be performed.

11. A method of infusing a medical fluid through a patient's venous catheter;
wherein the medical fluid is received through an infusion line that comprises an infusion tubing;
wherein the venous catheter comprises a catheter tubing, wherein the venous catheter is connected to the infusion line at a catheter coupling;
wherein the method comprises the steps of:
(a) having a catheter security device of claim 1:
(b) placing the catheter coupling onto the base piece;
(c) mating the cover piece to the base piece so that the catheter security device is in closed configuration;
(d) engaging the locking part and the locking counterpart to lock the catheter security device in closed configuration and form the enclosure;
wherein the catheter coupling is contained in the enclosure;
wherein the catheter tubing exits out of the enclosure at the first opening;
wherein the infusion tubing exits out of the enclosure at the second opening;
(e) administering the medical fluid through the infusion line and into the venous catheter while the catheter security device is locked;
(f) permanently breaking the locking mechanism and opening the enclosure to expose the catheter coupling.

12. The method of claim 11, further comprising stopping the administration of the medical fluid.

13. The method of claim 12, further comprising uncoupling the infusion line from the venous catheter.

14. The method of claim 11, wherein the first and second openings are sufficiently wide to avoid obstruction of the medical fluid infusion.

15. The method of claim 11, wherein the step of breaking of the locking mechanism is performed by cutting the locking mechanism.

16. The method of claim 11, wherein the step of breaking of the locking mechanism is performed by twisting off the locking mechanism.

17. The method of claim 11, wherein the enclosure has a length in the range of 5-15 cm and a width in the range of 1-5 cm.

18. The method of claim 11, wherein the catheter security device is a clamshell design with the cover piece being a first shell and the base piece being a second shell, and the device further comprises a hinged spine connecting the first shell and the second shell.

19. The method of claim 18, wherein the first shell comprises a first pair of notches and the second shell comprises a second pair of notches, wherein the first pair of notches and the second pair of notches are configured to match to form the openings for the enclosure.

20. The method of claim 11, wherein the catheter security device is a box design with the cover piece being a top lid and the base piece being a bottom box having a compartment, and wherein the top lid is configured to be fitted over the bottom box.
